(19) United States
(12) Patent Application Publication
(10) Pub. No.: US 2013/0143869 A1
(43) Pub. Date: Jun. 6, 2013

(54) USE OF A CATIONIC AMPHIPHILIC DRUG FOR THE PREPARATION OF A FORMULATION FOR THE REDUCTION OF SUBCUTANEOUS ADIPOSE TISSUE

(71) Applicants: Kevin Kiehm, Telgte (DE); Bernhard Hauptmeier, Gelnhausen (DE); Harry Abts, Oberursel (DE); Peter Boderke, Schwalbach (DE)

(72) Inventors: Kevin Kiehm, Telgte (DE); Bernhard Hauptmeier, Gelnhausen (DE); Harry Abts, Oberursel (DE); Peter Boderke, Schwalbach (DE)

(73) Assignee: Merz Pharma GmbH & CO. KGaA, Frankfurt am MAIN (DE)

(21) Appl. No.: 13/810,337

(22) PCT Filed: Jul. 14, 2011
(86) PCT No.: PCT/EP11/62103

§ 371 Date: Feb. 20, 2013

Related U.S. Application Data
(60) Provisional application No. 61/344,450, filed on Jul. 26, 2010.
(60) Provisional application No. 61/344,469, filed on Jul. 30, 2010.

(30) Foreign Application Priority Data
Jul. 16, 2010 (EP) 10169903.1

(51) Int. Cl. A61K 31/55; A61K 31/135; A61K 31/164; A61K 31/4706; A61K 31/343

(52) U.S. Cl. CPC A61K 31/55; A61K 31/4706; A61K 31/343; A61K 31/164; A61K 31/135
USPC 514/217; 514/313; 514/469; 514/620; 514/652

(57) ABSTRACT

Use of a cationic amphiphilic drug for the preparation of a formulation for the reduction and/or prevention of subcutaneous adipose tissue with improved effectiveness and better reproducible activity.

The present invention relates to the use of a cationic amphiphilic drug for the preparation of a formulation for the reduction of subcutaneous adipose tissue.

In general, cationic amphiphilic drugs are well known in the field of pharmaceutical active substances in the form of mostly oral antidepressants, antipsychotics, calcium canal blockers, beta receptor blockers, antihistaminics, and antifungals. Characteristically, cationic amphiphilic drugs contain a hydrophobic part consisting of a nonpolar ring system and a hydrophilic group with one or more nitrogen groups which can bear a net positive charge at physiological pH.

On the other hand, in the aesthetic and medical field subcutaneous adipose tissue is reduced by the injection of formulations containing aqueous liposome or micellar systems of phospholipids and/or bile acid or its derivatives.

However, those known aqueous liposome systems have the distinct disadvantage that their distribution inside the tissue is poor and thus the effect is fairly locally constricted to the immediate point of injection. Apart from that, it could be seen that the known active ingredients like phosphatidylcholin or desoxycholate can show side effects like localized inflammation and others which make the use of those systems for injection treatment uncomfortable. Furthermore deoxycholate is derived from animal-origin and thus possessing the risk of allergic reactions. Another disadvantage is the long tissue half-life of the compounds leading to persisting unwanted effects in the body.

Accordingly, up to date there is still the need for finding improved new active substances for the injection lipolysis which advantageously should also show a good reproducible activity.

Therefore, it is an object of the present invention to provide a formulation for a treatment directed to the reduction of adipose tissue that shows a equal or superior efficiency in reducing fatty tissue combined with a improved tolerability and safety profile as well as a good reproducible activity.

Surprisingly, it was found that the use of a cationic amphiphilic drug for the preparation of a formulation for the reduction and/or prevention of subcutaneous adipose tissue meets the object of the present invention.

The inventive use of the cationic amphiphilic drug of the present invention shows a better bioavailability and a better efficiency in reducing the fatty tissue and/or a better safety profile and/or a better tolerability than the known lipolysis systems comprising bile acid compounds, for example sodium deoxycholic acid, as active ingredients. Apart from that, it shows improved reactivity against subcutaneous localized fat cells while being able to reduce the side effects. Moreover, a consistent reproducibility, tolerability and safety are achieved by the fact that chemically defined drug compounds are used in contrast to animal derived material, e.g. deoxycholate.

It is thought that the advantageous effects are derived due to the ability of the amphiphilic drugs to interact with the cellular compounds in the target adipocytes which results in impairing membrane functions which dose dependently results in the accumulation of phospholipids within the targeted cells. This ability to impair the cell membrane function could be used to eliminate unwanted adipocytes in the subcutaneous fat tissue. However, as yet there is no proven link established in the relevant scientific literature between the tendency to induce phospholipidosis and the occurrence of cell toxicity.

The term “cationic amphiphilic drug” comprises drug compounds that have a hydrophobic part consisting of a nonpolar ring part and a hydrophilic group with one or more nitrogen containing groups which can bear a net positive charge at physiological pH.

The term “subcutaneous adipose tissue” comprises tissue in a layer that lies below the dermis of vertebrate skin, also called hypodermis. Under the term adipose tissue in particular any unwanted local fat deposits including simple unaesthetic appearances like cellulite and/or the following disease examples are understood. Under the term cellulite a condition causing topographic skin changes evident by skin dimpling is understood.

Lipomae are benign slow growing tumors of fat cells, preferred located in the subcutaneous fatty tissue that can occur in various forms and characteristics. They can build mucus, chalk and/or become ossified. Additionally, increased built of connective tissue and capsules can occur together with newly built blood vessels which are all classified as abnormal because the compression on the blood vessels as well as on the nerve cells is algetic. Lipomae occur in various syndromes like for example the Gardner syndrome, the Lanois-Bensaude syndrome, and the Proteus syndrome.

Lipomatosis dolorosa and cellulite are special forms of hypertrophic proliferation of fatty tissue which is located between the dermal fatty fascia and the underside of the dermis. Due to hormonal influences an enhanced capability to bind water in these fatty cells is observed which themselves initiate pressure and cause subsequently congestions in the lymphatic vessels. Additionally, compression and irritation to the peripheral sensitive nerves is applied so that the patients have an extreme sensitivity to contact. Over the years, irregular disseminated localised fatty nodes can built under the thinning dermis which are painful and show an unaesthetic character.

In this context also conditions like Lipoedema or lipodystrophic syndrome have to be mentioned.

The above addressed fatty tissue diseases demonstrate in contrast to alimentary related adipose disease pathophysiological tissue conditions that can be identified by histological scar and inflammation parameter as well as modifications in the histological fatty tissue morphology.

Under the term reduction it is in particular understood that the cytolysis of adipocytes and the degeneration of the prolific fatty tissue is reached.

The cationic amphiphilic drug can be applied topically. Examples for a topic application of a formulation comprising a cationic amphiphilic drug include a cream, a patch, a salve, a gel, a powder, a dressing, ointment, iontophoresis or transdermal system.

Alternatively, the cationic amphiphilic drug can be applied by subcutaneous injection. Examples for subcutaneous injection include aqueous solutions, suspensions, oily solutions, emulsions, microemulsions, liposomes, microspheres, nanoparticles and implants. The advantage of subcutaneous injections is the rapid onset of action and that the cytolytic effect is restricted to the targeted tissue. Furthermore, the systemic availability of compounds over time is reduced, since drug absorption from subcutaneous tissue is slow.

In a preferred embodiment of the present invention the cationic amphiphilic drug is a compound comprising a secondary of tertiary amino group (NRR′) which is linked via a linker sequence (L) with a chain length of one, two, three, four, five, six, seven, eight or nine atoms in the linker sequence backbone to an aromatic group (Ar) and which can be represented by the general formula RR′N-L-Ar.

The aromatic group (Ar) can be chosen from any aromatic group of one, two, three or four cyclic rings which can be carbon or heterocyclic rings like phenyl, naphthaline, and anthracene. The aromatic group (Ar) can comprise heterocyclic aromatic system such as pyridine, pyrimidine, imidazole, pyrrole, pyridazine, indole, phenothiazine, thiophene, dibenzodiazepine, thioxanthene, benzofurane, oxazole, indazole, purine, chinoline, oxepine, or dihydrodibenzthiepine.

The linker sequence (L) can be chosen from any divalent group having between one and nine chain atoms in the linker sequence backbone which can comprise carbon and hetero atoms like oxygen, sulphur or phosphor and which be substituted one, two, three or more times. Examples for a linker sequence of a compound used according to the present invention include alkyl, alkoxyl, alkylmercapto, alkylamino, alkylhydroxy, piperidyl, hydroxyl piperidyl, piperizinyl, alkylpiperidinyl, morpholinyl, tetrahydropyridinyl, pyrrolyl, cycloalkyl, piperazinyl, pyrazinyl, morpholinyl, alkyloxime.

The substituents R and R′ of the secondary or tertiary amino group RR′N can be either hydrogen in case there is a secondary amino group or they can be linear, branched or cyclic alkyl, alkenyl, or alkinyl, alkoxy, aryl, heteroaryl, or herterocyclyl.

More preferably, the cationic amphiphilic drug is chosen from the group consisting of Citaloprame in the enantiomeric R or S configuration as well as in racemic mixtures thereof, Fluoxetine Thirodazine, Promazine, Maprotiline, Loratadine, Imipramine, Doxepine, Desipramine, Clozapine, Clomipramine, Chlopromazine, Chloroquine, Labetalol, Dapoxetine, Fluvoxamine, Indalpine, Paroxetine, Zimelidine, Sertaline and Propanolol and salts, metabolites and prodrugs thereof.

Further examples of amphiphilic drugs that can be used according to the present invention include Fluphenazine, Haloperidol (Haldol, Serenace), Prochlorperazine, Mesoridazine, Loxapine, Molindone (Moban), Perphenazine (Trilifon), Thiothixene (Navane), Trifluoperazine (Stelazine), Fluphenazine (Prolixin), Droperidol, Zuclopenthixol (Clopixol), Periciazine, Triflupromazine, Olanzapine, Quetiapine, Asenapine, Sulpiride, Amisulpiride, Remoxipride, Melperone, Iloperidone, Paliperidone, Risperidone, Perospirone, Ziprasidone, Sertindole, Aripiprazole, Fluvoxamine (Luvox), Paroxetine (Paxil), Sertraline (Zoloft), Desvenlafaxine (Pristiq), Duloxetine (Cymbalta), Milnacipram (Ixel), Venlafaxine (Effexor), Mianserin (Tolvon), Mirtazapine, Atomoxetine (Strattera), Mazindol (Mazanor, Sanorex), Reboxetine (Edronax), Viloxazine (Vivalan), Bupropion, Tianeptine, Agomelatine, Amitriptyline (Elavil, Endep), Clomipramine (Anafranil), Doxepin (Adapin, Sinequan), Imipramine (Tofranil), Trimipramine (Surmontil), Nortriptyline (Pamelor, Aventyl), Protriptyline (Vivactil), Moclobemide (Aurorix, Manerix), Tranylcypromine (Parnate), Buspirone (Buspar), Gepirone (Ariza), Nefazodone (Serzone), Tandospirone (Sediel), Trazodone (Desyrel), Dosulepin, Etoperidone, Femoxetine, Lofepramine, Mazindol, Milnacipran, Nefazodone, Nisoxetine, Nomifensine, Oxaprotiline, Protryptiline, Viloxazine, Diphenhydramine, Loratadine, Desloratadine, Meclizine, Quetiapine, Fexofenadine Pheniramine, Cetirizine, Promethazine, Chlorpheniramine, Levocetirizine, Cimetidine, Famotidine, Ranitidine, Nizatidine, Roxatidine, Lafutidine, A-349,821, ABT-239, Ciproxifan Clobenpropit, Thioperamide, Thioperamide, JNJ 7777120, VUF-6002, Alprenolol, Bucindolol, Carteolol, Carvedilol, Labetalol, Nadolol, Penbutolol, Pindolol, Timolol, Acebutolol, Atenolol, Betaxolol, Bisoprolol, Celiprolol, Esmolol, Metoprolol, Nebivolol, and Butaxamine and salts, metabolites and prodrugs thereof.

In another preferred embodiment of the present invention the dose of cationic amphiphilic drug per one injection is between 50 μMol and 50 mMol.

According to another preferred embodiment of the present invention the formulation comprises at least one local anaesthetic agent. For example, lidocaine, procaine, tetracaine, etidocaine, mepivacaine, bupivacaine, prilocalne, and/or butanilicaine can be included as local anaesthetic agent in the mixture of the present invention.

According to another preferred embodiment of the present invention the formulation comprises at least one analgesic. The analgesic may be, but is not limited to, paracetamol, ibuprofen, diclofenac, naproxen, celecoxib, etoricoxib, lumiracoxib, parecoxib, rofecoxib, valdecoxib, nimesulid, oxicams, such as piroxicam, isoxicam, tonexicam, sudoxicam, and CP-14,304; the salicylates, such as salicylic acid, aspirin, disalcid, benorylate, trilisate, safapryn, solprin, diflunisal, and fendosal; the acetic acid derivatives, such as fenclofenac, indomethacin, sulindac, tolmetin, isoxepac, furofenac, tiopinac, zidometacin, acematacin, fentiazac, zomepiract, clidanac, oxepinac, and felbinac; the fenamates, such as mefenamic, meclofenamic, flufenamic, niflumic, and tolfenamic acids; the propionic acid derivates, such as benoxaprofen, flurbiprofen, ketoprofen, fenoprofen, fenbufen, indoprofen, pirprofen, carprofen, oxaprozin, pranoprofen, miroprofen, tioxaprofen, suprofen, alminoprofen, and tiaprofenic; and the pyrazoles, such as phenybutazone, oxyphenbutazone, feprazone, azapropazone, and trimethazone.

Additionally or alternatively, at least one complex builder, anti-foaming agent, and/or preservative can be comprised in a preferred embodiment of a formulation of the present invention. Examples for such additive substances like complex builders, anti-foaming agents, and/or preservatives are EDTA, dimethicone, phenol, cresol and its derivatives, benzoic acid, PHB ester, and/or sorbic acid.

The present invention can further comprise one or more pharmaceutical excipients selected from antioxidants, buffers, tonicity agents, hydrating agents, viscosity enhancers and/or viscosity modifiers, surface active agents, or a mixture thereof.

Antioxidants may be, but are not limited to, vitamin E, vitamin C, glutathione, coenzyme Q, resveratrol, bisulfite sodium, butylated hydroxyl anisole/toluene, cysteinate, dithionite sodium, gentisic acid, glutamate, formaldehyde sulfoxylate sodium, metabisulfite sodium, monothiogylcerol, propyl gallate, sulfite sodium, thiogycolate sodium, flavonoids, catalase, lycopene, carotenes, lutein, superoxide dismutase and peroxidises, or mixtures thereof.

Viscosity enhancers may be, but are not limited to, glycerol, xanthene gum, polyethylene glycol (PEG), alginate, carbomers, cellulose derivatives, dextrans, and carrageenan, starches, gum, acacia, tragacanth, gelatin, polyvinylpyrrolidone, albumin, dextran, or mixtures thereof.

Surface active agents may be, but are not limited to, polysorbate 20, polysorbate 80, polysorbate 40, polysorbate 60, polysorbate 65, Pluronic F68, Cetrimoniumbromid, Cetylpyridiniumchlorid, Brij 72, Brij 30, Brij 35, deoxycholate, lecithine, tocopheryl polyethylene glycol succinate or mixtures thereof.

According to the instant invention the formulation may comprise a medium in which the cationic amphiphilic drug is dissolved, dispersed or suspended. Said medium may be sterile water, phosphate-buffer saline (PBS), ringer solution, isotonic saline solution (0.9%), trometamol, citrate, carbonate, acetate, borate, amino acid, diethylamine, glucono delta lactone, glycine, lactate, maleic, methanesulfonic, monoethanolamine, tartrate buffer of choice, or any combination thereof.

The present invention also relates to a formulation comprising a cationic amphiphilic drug for use in a treatment for the reduction and/or prevention of subcutaneous adipose tissue.

Like that, a further increase in the adipocytolysis by the inventive formulation comprising a cationic amphiphilic drug can be achieved resulting in a shorter and more effective treatment. The treatment with a formulation of the present invention is preferably directed to cellulite tissue and/or local deposits of unaesthetic fatty tissue. In contrast to the known treatments especially the areas of mainly unaesthetic character are very receptive to the beneficial effect of a good biocompatibility and a good reproducibility.

In general, all unwanted and/or unaesthetic fatty tissue can be treated with the formulations of the present invention. This includes adipose tissue around the eyes, at the cheeks, in the neck and chin region, at the back, under and around the arms, at the thighs, in the upper and lower stomach region, at the knee, and/or so called lovehandles by males, gluteal bananas by females, and saddlebacks.

In particular, with the use of a cationic amphiphilic drug according to the present invention local deposits of unaesthetic fatty tissue around the eyes, under the arms, in the neck and chin region and/or at the tights are preferably treated. Those body regions often show a high sensitivity so that a possible reduction of side effects is most beneficial especially for the tissue in the mentioned regions.

The preparation of a formulation of the present invention can for instance be such that at least one cationic amphiphilic drug is mixed in water with physiologically acceptable salts. The preparation can be brought forward by any known form of preparation of aqueous mixtures.

Application of a formulation of the present invention carried out by any form of injection or topical application, in particular by subcutaneous injection.

In a preferred embodiment of the present invention the cationic amphiphilic drug is a compound comprising a secondary of tertiary amino group (NRR′) which is linked via a linker sequence (L) with a chain length of one, two, three, four, five, six, seven, eight or nine atoms in the linker sequence backbone to an aromatic group (Ar) and which can be represented by the general formula RR′N-L-Ar.

The aromatic group (Ar) can be chosen from any aromatic group of one to four cyclic rings like phenyl, naphthaline, and anthracene. The aromatic group (Ar) can also comprise heterocyclic aromatic system such as pyridine, pyrimidine, imidazole, pyrrole, pyridazine, indole, phenothiazine, thiophene, dibenzodiazepine, thioxanthene, benzofurane, oxazole, indazole, purine, chinoline, oxepine, or dihydrodibenzthiepine.

The linker sequence (L) can be chosen from any divalent group having between one and nine chain atoms in the linker sequence backbone which can comprise carbon and hetero atoms like oxygen, sulphur or phosphor and which be substituted one, two, three or more times. Examples for a linker sequence of a compound used according to the present invention include alkyl, alkoxyl, alkylmercapto, alkylamino, alkylhydroxy, piperidyl, hydroxyl piperidyl, piperizinyl, alkylpiperidinyl, morpholinyl, tetrahydropyridinyl, pyrrolyl, cycloalkyl, piperazinyl, pyrazinyl, morpholinyl, alkyloxime.

The substituents R and R′ of the secondary or tertiary amino group RR′N can be either hydrogen in case there is a secondary amino group or they can be linear, branched or cyclic alkyl, alkenyl, or alkinyl, alkoxy, aryl, heteroaryl, or herterocyclyl.

More preferably, the cationic amphiphilic drug is chosen from the group consisting of Citaloprame in the enantiomeric R or S configuration as well as in racemic mixtures thereof, Fluoxetine Thirodazine, Promazine, Maprotiline, Loratadine, Imipramine, Doxepine, Desipramine, Clozapine, Clomipramine, Chlopromazine, Chloroquine, Labetalol, Dapoxetine, Fluvoxamine, Indalpine, Paroxetine, Zimelidine, Sertaline and Propanolol and salts, metabolites and prodrugs thereof.

Further examples of suitable cationic amphiphilic drugs include the drugs stated above like Fluphenazine, Haloperidol (Haldol, Serenace), Prochlorperazine, Mesoridazine, Loxapine, Molindone (Moban), Perphenazine (Trilifon), Thiothixene (Navane), Trifluoperazine (Stelazine), Fluphenazine (Prolixin), Droperidol, Zuclopenthixol (Clopixol), Periciazine, Triflupromazine, Olanzapine, Quetiapine, Asenapine, Sulpiride, Amisulpiride, Remoxipride, Melperone, Iloperidone, Paliperidone, Risperidone, Perospirone, Ziprasidone, Sertindole, Aripiprazole, Fluvoxamine (Luvox), Paroxetine (Paxil), Sertraline (Zoloft), Desvenlafaxine (Pristiq), Duloxetine (Cymbalta), Milnacipram (Ixel), Venlafaxine (Effexor), Mianserin (Tolvon), Mirtazapine, Atomoxetine (Strattera), Mazindol (Mazanor, Sanorex), Reboxetine (Edronax), Viloxazine (Vivalan), Bupropion, Tianeptine, Agomelatine, Amitriptyline (Elavil, Endep), Clomipramine (Anafranil), Doxepin (Adapin, Sinequan), Imipramine (Tofranil), Trimipramine (Surmontil), Nortriptyline (Pamelor, Aventyl), Protriptyline (Vivactil), Moclobemide (Aurorix, Manerix), Tranylcypromine (Parnate), Buspirone (Buspar), Gepirone (Ariza), Nefazodone (Serzone), Tandospirone (Sediel), Trazodone (Desyrel), Dosulepin, Etoperidone, Femoxetine, Lofepramine, Mazindol, Milnacipran, Nefazodone, Nisoxetine, Nomifensine, Oxaprotiline, Protryptiline, Viloxazine, Diphenhydramine, Loratadine, Desloratadine, Meclizine, Quetiapine, Fexofenadine Pheniramine, Cetirizine, Promethazine, Chlorpheniramine, Levocetirizine, Cimetidine, Famotidine, Ranitidine, Nizatidine, Roxatidine, Lafutidine, A-349,821, ABT-239, Ciproxifan Clobenpropit, Thioperamide, Thioperamide, JNJ 7777120, VUF-6002, Alprenolol, Bucindolol, Carteolol, Carvedilol, Labetalol, Nadolol, Penbutolol, Pindolol, Timolol, Acebutolol, Atenolol, Betaxolol, Bisoprolol, Celiprolol, Esmolol, Metoprolol, Nebivolol, and Butaxamine and salts, metabolites and prodrugs thereof.

According to a preferred embodiment of the present invention the formulation is a topic formulation. Examples of such a topic formulation include include a cream, a patch, a salve, a gel, a powder, a dressing, ointment, iontophoresis or transdermal system.

Alternatively, the formulation is a subcutaneous injection. Examples for subcutaneous injection include aqueous solutions, suspensions, oily solutions, emulsions, microemulsions, liposomes, microspheres, nanoparticles and implants. The advantage of subcutaneous injections is the rapid onset of action and that the cytolytic effect is restricted to the targeted tissue. Furthermore, the systemic availability of compounds over time is reduced, since drug absorption from subcutaneous tissue is slow.

Preferably, the formulation is intended for a treatment which is directed to cellulite tissue and/or local deposits of unaestethic or unwanted fatty tissue.

Local deposits of unseasthetic fatty tissue around the eyes, under the arms, in the neck and chin region and/or at the thights can preferably be treated.

In a preferred embodiment of the present invention the dose per one injection is preferably between 50 μMol and 50 mMol.

Preferably, each injection unit of the formulation has a distinct dose of the cationic amphiphilic drug according to the invention. This dose can reach from about 50 μMol to about 50 mMol, preferred from about 100 μMol to about 10 mMol, per volume of one injection shot. One injection shot is sized from about 0.15 ml of the formulation to about 2.0 ml, more preferable between 0.5 and 1 ml.

For the treatment of an adult patient by application of injection solutions the above mentioned doses per volume of one injection shot dependant of the size of the fatty tissue to be treated are administered.

The dose is also dependant to the size of the fat depot and/or the disordered distribution of the fat cells and/or the type of adipose disease. It should be tailored to the needs of the single patient.

In another embodiment the object of the present invention is also solved by the use of Ketoconazloe as active ingredient in a formulation for a treatment in the reduction and/or prevention of subcutaneous adipose tissue.

Ketoconazole can be subsumed under the general formula RR′N-L-Ar given above as there is a hydrophobic end with phenyl and imidazole rings. But the more hydrophilic group is comprised in the linker sequence L with a dioxolane part. Thus, Ketoconazole is not seen as a member of the group of cationic amphiphilic drugs.

### EXAMPLES

### Example 1

### Metabolic Activity and Membrane Integrity of Human Adipocytes

The effects on cytotoxicity in human adipocyte cells of 8 compounds were tested with reference codes:

### 1 Chloroquine

### 2 Citalopram HBr

3 Escitalopram oxalate

### 4 Fluoxetine HCl

### 5 Ketoconazole

### 6 Diltiazem HCl

### 7 Labetalol HCl

8 Deoxycholate Na (reference)

Metabolic activity was selected as relevant primary biological cytotoxicity endpoint. In addition the membrane integrity was determined by measuring the release of LDH from the cells (LDH assay).

### EXPERIMENTAL PROCEDURES

### Internal Reference Items

### Doxorubicin

### Camptothecin

### Cells

Human white preadipocytes (HWP)

### Chemicals, Media, Additives

| | |
|---|---|
| Apoptosis detection kit | BD Pharmingen |
| Cytotoxicity detection kit (LDH) | Roche |
| DMSO | Roth |
| Nile Red | Sigma |
| Pen/Strep | ccPro |
| Propidium iodide | Sigma |
| Resazurin | Sigma |
| Triton-x 100 | VWR |
| Trypan blue | Sigma |

### Media

| | |
|---|---|
| Pre-adipocyte growth medium | PromoCell |
| Pre-adipocyte differentiation medium | PromoCell |
| Adipocyte nutrition medium | PromoCell |

All media were free of serum; for differentiation and cytotoxicity experiments 0.5% Pen/Strep was added to the media.

### Solutions

Resazurin

| | |
|---|---|
| Stock solution | 4.5 mM resazurin in PBS |
| Working solution | 1:100 dilution of stock in PBS |

### Differentiation of Human White Preadipocytes (HWP)

HWP were obtained in passage two and differentiated in vitro to mature adipocytes. Briefly, cells were initially cultured in preadipocyte growth medium at 37° C. and 5% CO₂ to 70-80% confluency. Then, cells were seeded at 40,000 cells/ml into 96-well flat bottom tissue culture plates and incubated overnight to allow attachment. Differentiation was initiated by changing the medium to preadipopcyte differentiation medium for an additional 72 hrs. After differentiation was induced, the medium was replaced by adipocyte nutrition medium that was refreshed every 2-3 days over a period of 14 days until termination of differentiation. Differentiation status was checked via nile red staining of control cells. Successful adipocyte differentiation was observed by fluorescently stained lipid droplets in the cytoplasm.

### Preparation of Test Items and Reference Items

The test items 1 to 8 were prepared according to Table 1 to yield respective stock solutions. Said stock solutions were further diluted in assay media to obtain the double concentrated working solutions, which were subsequently diluted 1:2 in the final assay. The final Cₘₐₓ in the cytotoxicity assay was 1 mM with the exception of Ketoconazole which was used at 500 μM (due to generation of insoluble precipitates at higher concentrations). Deoxycholate Na was selected as “reference compound”.

All experiments were carried out with a maximal solvent concentration of 1% (v/v). The negative control wells contained media only plus the respective solvent. Unused portions of the stock- and working solutions were stored at −20° C.

TABLE 1
Test item solutions

| | | Amount | | | Solvent | |
|---|---|---|---|---|---|---|
| | | Weighed | MW | | Added | Final |
| No. | Name | [mg] | [Da] | Solvent | [μl] | [mM] |
| 1 | Chloroquine | 4.2 | 515.87 | PBS | 81.4 | 100 |
| | disphosphate | | | | | |
| 2 | Citalopram HBr | 3.8 | 405.31 | 50% | 94.5 | 100 |
| | | | | DMSO | | |
| 3 | Escitalopram | 3.6 | 414.42 | 50% | 87.4 | 100 |
| | oxalate | | | DMSO | | |
| 4 | Fluoxetine HCl | 3.0 | 345.8 | 50% | 89.4 | 100 |
| | | | | DMSO | | |
| 5 | Ketoconazole | 3.1 | 531.44 | DMSO | 118.9 | 50 |
| 6 | Diltiazem HCl | 3.0 | 415.0 | PBS | 72.3 | 100 |
| 7 | Labetalol HCl | 3.2 | 364.87 | 50% | 90.4 | 100 |
| | | | | DMSO | | |
| 8 | Deoxycholate | 3.1 | 414.55 | PBS | 75.5 | 100 |
| | Na | | | | | |

### Experimental Setup Cytotoxicity Assay

For the cytotoxicity assays in this study pre-adipocytes were seeded at 40,000 cells/ml into a 96-well microtiter flat bottom plate. The test items were prepared in separate plates starting with the double concentrated maximal test concentration followed by a 1:2 serial dilution (Chloroquine and Fluoxetine 1:3) and then added to the cells. The test items were tested at 6 replicates/concentration. Each plate included negative controls (media plus solvent, 6 replicates), a drug interference control (all concentrations, duplicates), a background control (14 replicates), a positive (=cytotoxic) control (6 replicates doxorubicin, 1 μM final) and a positive control for maximal LDH-release (2 replicates, 2% Triton-x 100 final).

Relative cell viability was determined after 48 hrs exposure based on metabolic activity (i.e. resazurin conversion). Using a Victor³ 1420 Multilabel Counter, the resazurin conversion to the fluorescent resorufin by metabolically active cells was recorded at 560_{Ex}/590_{Em}. Membrane integrity was measured after 6 hrs and 48 hrs and the enzymatic activity of released LDH in the cell supernatant after 30 min of incubation at 492 nm_{absorption}/690 nm_{reference} by a spectrophotometer. The volume withdrawn from the cultures for the LDH-assay was not replaced to keep cells in a less disturbed environment. The assay was conducted in a multiplexed format.

### Data Analysis LDH Assay

For the dose-response relationship, absolute absorption (OD treated wells−background) was plotted against the test item concentrations representing LDH leakage from the cells. For a better estimation of the test items' potential influencing the membrane integrity, relative, maximal LDH release in percent (see Table 2) was also calculated according to the following formula:

Relative maximal LDH release=[Highest compound induced LDH release/Triton-x 100 maximal LDH release*100]. Results are given in Table 2.

### IC₅₀ Calculation:

In order to derive meaningful IC₅₀ values from the primary data, the entire biological window of activity ranging from 0% to 100% viability should be covered and the resolution has to be sufficient. To improve data quality, the serial dilution factor used in previous experiments was reduced to a 1:2 and 1:3 dilution. An IC₅₀ calculation is not possible for compounds that do not exert a cytotoxic potential covering the whole biological window. IC₅₀ (half maximal inhibitory concentration, calculated via Sigma Plot 10) values were determined for each test substance 1 to 8.

TABLE 2
Summary IC 50 values (metabolic activity after 48 hrs) and
maximal LDH-release

| | | Rel. max. |
|---|---|---|
| Test substance | | LDH release |
| no. | IC₅₀/[μM] | [%] 48 hrs |
| 1 | 599 | 101.8 |
| 2 | 431 | 100.7 |
| 3 | 491 | 88.0 |
| 4 | 52 | 101.0 |
| 5 | 78 | 92.2 |
| 6 | 800 | 94.5 |
| 7 | 659 | 101.1 |
| 8 (reference) | 446 | 98.0 |

### Example 2

### Metabolic Activity and Membrane Integrity of Human Adipocytes

The effects on cytotoxicity in human adipocyte cells of a further 9 compounds were tested. The experimental set-up was identical to the one in example 1.

### Results

TABLE 3

IC₅₀ [μM]

| TEST ITEM | 48 hrs LDH | 48 hrs Resazurin |
|---|---|---|
| Trifluoperazine | ≈14.3 | 17.4 |
| Fluphenazine | ≈20.6 | ≈25.5 |
| Paroxetine | ≈31.2 | 39.3 |
| Perphenazine | ≈20.3 | 25 |
| Clomipramine | ≈39.1 | 52.2 |
| Desloratadine | ≈41 | ≈92.4 |
| Carvedilol | ≈22.2 | 28.4 |
| Duloxetine | ≈31.9 | 42.9 |
| Thioconazole | ≈7.4 | 10.5 |
| Deoxycholate | ≈379 | ≈616 |

Compared to example 1 deoxycholate shows here a lower efficacy in elimination of adipocytes (IC 50 value of 616 μM (example 2) v. 446 μM (example 1). This indicates that the elimination effect on adipocytes by deoxycholate shows some variability and that identified new compounds are effective even under conditions were deoxycholate is less efficient.

### Example 3

### Apoptosis/Necrosis Detection

### Background

The apoptotic program is characterized by certain morphologic features. Loss of plasma membrane asymmetry is a well established indicator for apoptotic cells. In apoptotic cells, the membrane phospholipid phosphatidylserine (PS) is translocated from the inner to the outer leaflet of the plasma membrane, thereby exposing PS to the external environment. In general, this PS translocation is preceded by caspase activation. Annexin V labelled with FITC is a Ca²⁺ dependent phospholipid-binding protein with high affinity for PS and binds to cells with externalized PS. Cells stained with Annexin V*FITC are therefore considered in an early apoptotic stage.

### Assay System

Staining with Annexin V-FITC is typically used in conjunction with a vital dye such as propidium iodide (PI) to allow discrimination between early apoptotic and necrotic cells. Necrotic dying cells are PI positive only. Cells that are viable are Annexin V and PI negative; cells that entered apoptosis are Annexin V positive and PI negative and cells that are in post apoptotic and/or necrotic phase are both Annexin V and PI positive.

### Experimental Set-Up

Human white adipocytes that had been differentiated in vitro from pre-adipocytes as describe above were used. Measurements were performed after 5 hrs to catch early apoptotic/necrotic events and after 24 hrs and 48 hrs for later stages. Naringenin at 500 μM (reported to induce apoptosis in adipocytes) was used as positive control and media plus solvent was used as negative control. Each compound was tested in three different concentrations. The middle concentration was selected to roughly equal the IC₅₀ determined in Example 1. Two more concentrations, one below and one above the IC₅₀ were added. This made sure that the tested concentration range had biological significance.

### Flow Cytometry

Flow cytometry was performed with a dual laser FC500. After completion of the staining procedure, tubes were grouped into a FC500 carousel for semi-automated data acquisition. Cells were first acquired in “Setup” mode to tune the parameters FS, SS, FL1 and FL2. After completion of the setup, baseline compensation was switched on and cells were acquired. Cells were acquired without life-gate but using FS threshold to exclude debris. All cells were acquired using individually corrected settings.

### Analysis

After completion of data acquisition, analysis was done using the CXP software. If necessary, software-based post acquisition compensation was performed to fine-correct for fluorescence overlap not adjusted for during the acquisition phase. Quadrant markers were set based on the negative controls.

### Results

In table 4, relative frequency of human adipocytes in healthy stage, early apoptotic stage and late apoptotic/necrotic stage are listed with the corresponding exposure times and test item concentrations.

TABLE 4

| | | Incubation | | Early | Necrosis/ | |
|---|---|---|---|---|---|---|
| | Concentration | Time | Normal | Apoptosis | Late Apoptosis | |
| Test Item | [μM] | [hrs] | [A-V*/PI] | [A-V*/PI*] | [A-V*/PI*] | Total |
| Neg. Control | n.a. | 5 | 95.2 | 3.8 | 0 | 100 |
| | n.a. | 24 | 92.2 | 7.6 | 0.2 | 100 |
| | n.a. | 48 | 95 | 2.3 | 2.7 | 100 |
| Naringenin | 500 | 5 | 83.2 | 16 | 0.8 | 100 |
| | 500 | 24 | 81.8 | 14.1 | 4.1 | 100 |
| | 500 | 48 | 81.2 | 12.1 | 5.7 | 100 |
| Fluoxetine | 40 | 5 | 73.1 | 26.9 | 0 | 100 |
| | 60 | 5 | 45.2 | 58.5 | 1.3 | 100 |
| | 80 | 5 | 47 | 51.4 | 1.6 | 100 |
| Fluoxetine | 40 | 24 | 49.9 | 47.5 | 2.6 | 100 |
| | 60 | 24 | 14.7 | 78.7 | 5.5 | 100 |
| | 80 | 24 | 14 | 41.9 | 44.1 | 100 |
| Fluoxetine | 40 | 48 | 69.6 | 22.7 | 7.7 | 100 |
| | 60 | 48 | 42.3 | 37.9 | 19.8 | 100 |
| | 80 | 48 | 2.7 | 55.1 | 42.2 | 100 |
| Ketoconazole | 60 | 5 | 72.1 | 26.5 | 1.4 | 100 |
| | 80 | 5 | 63.8 | 34 | 2.2 | 100 |
| | 100 | 5 | 73.3 | 23.6 | 3.1 | 100 |
| Ketoconazole | 60 | 24 | 77.9 | 19.5 | 2.6 | 100 |
| | 80 | 24 | 59.4 | 22.7 | 17.9 | 100 |
| | 100 | 24 | 0.5 | 16.9 | 82.6 | 100 |
| Ketoconazole | 60 | 48 | 87.1 | 9.1 | 3.8 | 100 |
| | 80 | 48 | 36.6 | 18.5 | 44.9 | 100 |
| | 100 | 48 | 0.5 | 36.9 | 52.5 | 100 |
| Citalopram | 250 | 5 | 77.9 | 21.5 | 0.6 | 100 |
| | 350 | 5 | 67.2 | 31.3 | 1.5 | 100 |
| | 450 | 5 | 68.4 | 30.5 | 1.1 | 100 |
| Citalopram | 250 | 24 | 65.4 | 32.4 | 2.2 | 100 |
| | 350 | 24 | 37.7 | 57.6 | 4.7 | 100 |
| | 450 | 24 | 50.1 | 26 | 23.9 | 100 |
| Citalopram | 250 | 48 | 79.6 | 23.1 | 3.3 | 100 |
| | 350 | 48 | 53.7 | 35.6 | 10.7 | 100 |
| | 450 | 48 | 29.1 | 44.9 | 26 | 100 |

It can be seen that Naringenin, a compound that has been reported in the literature to induce apoptosis in human adipocytes, shows a small but clear increase in apoptotic/necrotic adipocytes over time.

Fluoxetine, on the other hand, which was used at 40, 60, and 80 μM, rapidly induced strong apoptosis. After only 5 h with 80 μM fluoxetine more than 50% of cells are apoptotic. Thus fast and strong apoptosis induction can be considered primary mode of cell death.

Citalopram, which was used at 250, 350 and 450 μM shows clear apoptosis after 5 hrs with virtually no necrosis. After 24 hrs exposure, apoptosis is still very present but first shifts towards necrotic/late apoptotic cells can be seen at 450 μM.

Ketoconazole caused apoptosis after 5 hrs at 60, 80, and 100 μM increasing over time. Cell death at 100 μM occurs very rapid and necrosis starts to become visible after 48 hrs exposure and at 80 μM. Apoptosis can be considered primary mode of cell death at the lower concentration range with necrosis taking place later at higher concentrations.

### Example 4

### In Vivo Elimination of Adipocytes by Fluoxetine in a Animal Model

The study was designed to provide data about tissue effects after repeated subcutaneous injection for fat dissolution in rats. This study was performed in rats, which is the preferred species for this type of local tolerance study.

Although quantification of eliminated fat-cells by conventional histology is difficult, the secondary inflammation seen after disruption of adiopcytes may be used as surrogate-marker for the compound induced adipocytolysis.

### Materials and Methods

### Fluoxetine

Name: Fluoxetine

Dosage: 1 mg/mL

Physical Appearance Aqueous formulation

### Vehicle

Name: Placebo-Muster (buffer only)

Physical Appearance Aqueous formulation

### Animals

Species/strain: Healthy rats, Crl:CD (SD) rats (Full-Barrier)

Sex: male

Weight at commencement of the study: approximately 400 g

Prior to the first administration a detailed clinical observation was made of all animals.

Animals were individually marked at the tail using a permanent marker.

10 animals were divided into 2 groups.

TABLE 5
Overview groups/Test Item/Concentration

| | Test Item/Concentration |
|---|---|
| Group-No. | (Volume of application) |
| 1 | Fluoxetine/300 μL |
| 2 | Vehicle/300 μL |

Animals were administered 3 times subcutaneously into the left abdominal fat tissue (Day 1, Day 7 and Day 28) with 300 μL of Fluoxetine or vehicle (see Table 1). The right abdominal fat tissue of each animal remained untreated.

On day 29 the animals were anaesthetized and euthanized and the entire abdominal skin with adherent subcutaneous fat was peeled off using tweezers and sharp scissors. Two fat biopsies were taken from each animal (fixation on cork in formalin): treated left abdominal fat tissue and untreated right abdominal fat tissue.

Throughout the 29-day observation period, the weight gain of the animals was within the expected range.

### Clinical Examination

A careful clinical examination was made at least once a day for the period of observation.

General clinical observation was made preferably at the same time each day and considering the peak period of anticipated effects after dosing. The health condition of the animals was recorded.

General clinical observation included changes in the skin and fur, eyes and mucous membranes. Also respiratory, circulatory, autonomic and central nervous systems and somatomotor activity and behavior pattern were examined. Particular attention was directed to observations of tremor, convulsions, salivation, diarrhoea, lethargy, sleep and coma. Individual reactions of each animal were recorded at each observation time.

### Histopathology

Two cuts inside the application area, one cut outside the application area and one cut through the native side was performed.

Overall changes in fat-tissue size (macroscopic/low magnification), effects on adipocytes (necrosis/apoptosis of cells, size of cells—semi-quantitative compared to native control site, number of cells (semi-quantitative compared to native control side) and inflammatory processes (type and number of infiltrating cells (semi-quantitative compared to native control side) were evaluated histological.

### Results

### Clinical Observation

No clinical signs of systemic toxicity were observed during the treatment period in any of the animals.

### Histopathology

Histopathological assessment revealed the following findings:

### Untreated Skin/Subcutis (Right Body Side)

The untreated skin/subcutis was generally free of inflammatory infiltrates. Tissue samples showed only rarely minimal infiltrates of mononuclear cells, in one animal together with minimal infiltrates of granulocytes.

### Administration Sites of Fluoxetine

Changes noted at administration sites of Fluoxetine were generally observed in the subcutaneous connective tissue and/or adipose tissue, were minimal or mild in degree and mainly comprised: minimal or mild edema, minimal or mild haemorrhage, minimal to moderate infiltrates of mononuclear cells, minimal infiltrates of granulocytes, minimal single cell death of inflammatory cells and/or connective tissue and minimal or mild fibrosis.

In the peripheral sections of administration sites, histological changes were less frequent, but corresponded generally to the type of changes seen in central sections.

Overall, a distinct inflammatory reaction was observed after repetitive injection of fluoxetine. As this inflammatory reaction can be taken as surrogate-marker for adipocytolysis, it can be concluded that administration of fluoxetine causes adipocytolysis.

### Administration Sites of Vehicle

At administration sites of the vehicle, histological changes noted were mostly minimal and seen in a low number of samples. These were mainly minimal or mild haemorrhages, minimal infiltrates of mononuclear cells or granulocytes, minimal single cell death of inflammatory cells. Together, these minor lesions were considered to be indicative of effects of the vehicle itself, possibly together with local trauma caused by the technical procedure of repetitive subcutaneous injection. Histological changes noted at administration sites of the vehicle were least prominent.

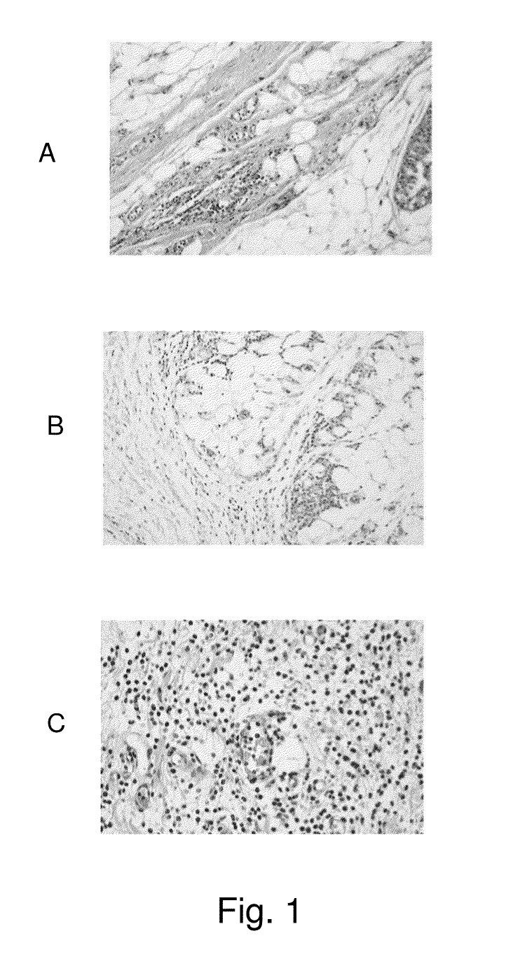

FIG. 1 shows the secondary inflammation seen after disruption of adipocytes, which was used as surrogate-marker for the compound induced adipocytolysis.

FIG. 1A (magnification of 20×) shows that the negative control (vehicle) only cause minimal secondary inflammation mainly as result of technical procedure of repetitive injection

FIG. 1B (magnification of 20×) and C (magnification of 40×) show that fluoxetine causes an inflammatory reaction in the subcutaneous fat-tissue. In FIG. 1C the nuclei of the infiltrated lymphocytes show up in dark.

### CONCLUSION

The histology-data as represented by FIG. 1 show clearly the inflammatory reaction after repetitive injection of fluoxetine into the subcutaneous fat-tissue. As this inflammatory reaction can be taken as surrogate-marker for adipocytolysis, it can be concluded that administration of fluoxetine causes adipocytolysis.

1-17. (canceled)

18. A method for the reduction and/or prevention of subcutaneous adipose tissue comprising administering to a human or animal subject, a formulation comprising a cationic amphiphilic drug in an amount effective for the reduction and/or prevention of subcutaneous adipose tissue.

19. The method of claim 18, wherein the cationic amphiphilic drug is administered topically.

20. The method of claim 18, wherein the cationic amphiphilic drug is administered by subcutaneous injection.

21. The method of claim 18, wherein the subcutaneous adipose tissue comprises cellulite tissue and/or local deposits of unaesthetic fatty tissue.

22. The method of claim 18, wherein the cationic amphiphilic drug is a compound comprising a secondary or tertiary amino group (NRR′) which is linked via a linker sequence (L) with a chain length of one, two, three, four, five, six, seven, eight or nine atoms in the linker sequence backbone to an aromatic group (Ar) and which can be represented by the general formula RR′N-L-Ar.

23. The method of claim 18, wherein the cationic amphiphilic drug is selected from the group consisting of Citalopram, Fluoxetine, Chloroquine, Labetalol and Propanolol.

24. The method of claim 18, wherein the cationic amphiphilic drug is selected from the group consisting of Thirodazine, Promazine, Maprotiline, Loratadine, Imipramine, Doxepine, Desipramine, Clozapine, Clomipramine, Chlopromazine, Dapoxetine, Fluvoxamine, Indalpine, Paroxetine, Zimelidine, and Sertaline.

25. The method of claim 18, wherein the dose of cationic amphiphilic drug per one injection is between 50 μMol and 50 mMol.

26. The method of claim 18, wherein the formulation comprises at least one local anaesthetic agent.

27. A pharmaceutical composition comprising a cationic amphiphilic drug for administration to a subject in a treatment for the reduction and/or prevention of subcutaneous adipose tissue.

28. The pharmaceutical composition of claim 27, wherein the cationic amphiphilic drug is a compound comprising a secondary or tertiary amino group (NRR′) which is linked via a linker sequence (L) with a chain length of one, two, three, four, five, six, seven, eight or nine atoms in the linker sequence backbone to an aromatic group (Ar) and which can be represented by the general formula RR′N-L-Ar.

29. The pharmaceutical composition of claim 27, wherein the cationic amphiphilic drug is selected from the group consisting of Citalopram, Fluoxetine Chloroquine, Labetalol and Propanolol.

30. The pharmaceutical composition of claim 27, wherein the cationic amphiphilic drug is selected from the group consisting of Thirodazine, Promazine, Maprotiline, Loratadine, Imipramine, Doxepine, Desipramine, Clozapine, Clomipramine, and Chlopromazine.

31. The pharmaceutical composition of claim 27, which is a topic formulation.

32. The pharmaceutical composition of claim 27, which is in a form for subcutaneous injection.

33. The pharmaceutical composition of claim 27, wherein the treatment is directed to cellulite tissue and/or local deposits of unaesthetic fatty tissue.

34. The pharmaceutical composition of claim 33, wherein the local deposits of unaesthetic fatty tissue are located around the eyes, under the arms, in the neck and chin region and/or at the thighs.
